(19) United States
(12) Patent Application Publication
(10) Pub. No.: US 2026/0041517 A1
(43) Pub. Date: Feb. 12, 2026

(54) Enhanced Imaging Head Stabilization Device

(71) Applicant: pro med instruments GmbH, Freiburg Im Breisgau (DE)

(72) Inventors: Christian Schlenk, Oberkirch (DE); Andreas Blum, Ehrenkirchen (DE); Simon-Martin Höfer, Appenweier (DE); René Patrick Machts, Emmendingen (DE); Marco Willesch, Eichstetten (DE); Matthias Esser, Feiburg (DE); Severin Moosmann, Friesenheim (DE); Matthias E. Schuele, Feiburg (DE)

(21) Appl. No.: 19/100,528

(22) PCT Filed: Aug. 4, 2022
(86) PCT No.: PCT/IB2022/000447

§ 371 Date: Jan. 31, 2025

(51) Int. Cl. A61B 90/14

(52) U.S. Cl. CPC A61B 90/14

(57) ABSTRACT

A head stabilization device useable for stabilizing a head of a patient during a medical procedure includes a tensioning assembly operable to increase or decrease an amount of force the stabilization assembly applies to the patient's head. The head stabilization device includes an adjustment feature to adjust a spacing between frame members. The head stabilization device further includes features that promote enhanced imaging output when the head stabilization device is used during imaging.

### BACKGROUND

During certain medical procedures it may be necessary or desirable to stabilize all or a portion of a patient such that the patient or portion of the patient is immobilized. In certain neurological procedures the portion stabilized may include the head and/or neck of the patient. Certain devices and methods may be used to stabilize a certain portion of the patient. For example, a skull clamp is a type of head stabilization device that may be used to stabilize the head and/or neck of the patient. Furthermore, it may also be necessary or desirable to use various imaging modalities to obtain images of the patient before, during, and/or after a procedure. In certain circumstances such imaging occurs when the patient is stabilized using a head stabilization device. While a variety of head stabilization devices and method of use of the same have been made and used, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements.

FIG. 1 depicts a front perspective view of an exemplary skull clamp.

FIG. 2 depicts a rear perspective view of the skull clamp of FIG. 1.

FIG. 3 depicts a bottom view of the skull clamp of FIG. 1.

FIG. 4 depicts a partial top cross section view of the skull clamp of FIG. 1, showing a lock release feature.

FIG. 5A depicts a partial front cross section view of the skull clamp of FIG. 1, showing the skull clamp in a locked state where the width of the frame cannot be increased.

FIG. 5B depicts a cross section view of the skull clamp of FIG. 5A taken along line 5B-5B of FIG. 5A.

FIG. 6A depicts a partial front cross section view of the skull clamp of FIG. 1, showing the skull clamp in an unlocked state where the width of the frame can be increased.

FIG. 6B depicts a cross section view of the skull clamp of FIG. 6A taken along line 6B-6B of FIG. 6A.

FIG. 7 depicts a cross section view of the skull clamp of FIG. 1 taken along line 7-7 of FIG. 1.

FIG. 8 depicts a cross section view of the skull clamp of FIG. 1 taken along line 8-8 of FIG. 1.

FIG. 9 depicts a cross section view of the skull clamp of FIG. 1 taken along line 9-9 of FIG. 1.

FIG. 10A depicts a partial front view of the skull clamp of FIG. 1, showing the pinning force set to a starting or initial state.

FIG. 10B depicts a cross section view of the skull clamp of FIG. 10A.

FIG. 10C depicts a cross section view of the skull clamp of FIG. 10A, taken along line 10C-10C of FIG. 10A.

FIG. 10D depicts a cross section view of the skull clamp of FIG. 10A, taken along line 10D-10D of FIG. 10A.

FIG. 11A depicts a partial front view of the skull clamp of FIG. 1 after increasing the pinning force from the starting or initial state.

FIG. 11B depicts a cross section view of the skull clamp of FIG. 11A.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

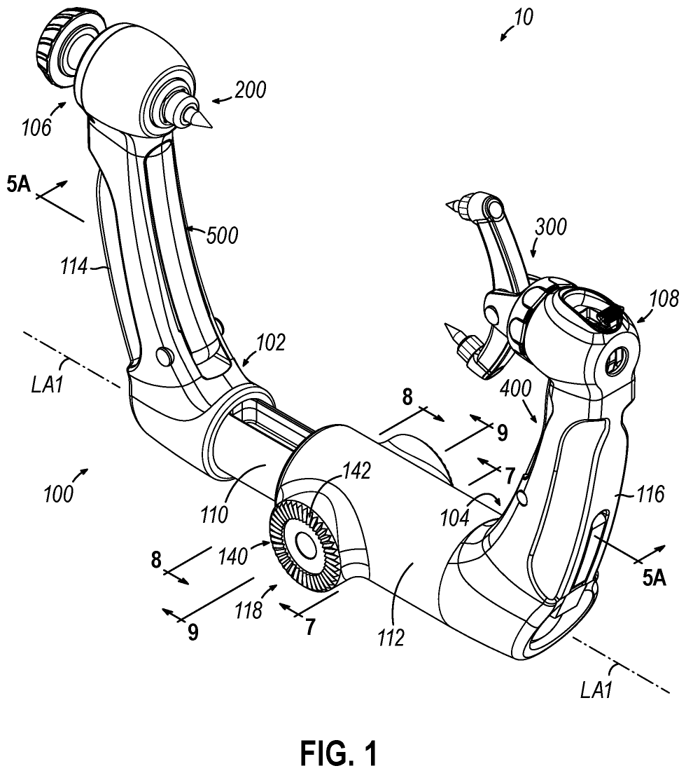

FIG. 1 illustrates an exemplary head stabilization or fixation device. Throughout the specification the term “HFD” is used interchangeably with the terms “head stabilization device,” “head fixation device,” or “skull clamp.” In the illustrated versions here HFD has the shape or form of a skull clamp (10). In this respect, skull clamp (10) comprises a frame (100). Frame (100) includes a first frame portion (102) and a second frame portion (104). Frame portions (102, 104) include respective receiving portions (106, 108) that are configured to receive a stabilization assembly. In the illustrated version, stabilization assembly (200) is received by receiving portion (106) of frame portion (102). Furthermore, stabilization assembly (300) is received by receiving portion (108) of frame portion (104).

Skull clamp (10) includes a frame adjustment feature (400) that is operable to adjust the relative spacing between frame portions (102, 104). Additional components and operability of frame adjustment feature (400) will be discussed in detail below. Skull clamp (10) also includes a tensioning assembly (500) that is operable to adjust an amount of force a stabilization assembly applies to a stabilizing feature contacting the patient. Additional components and operability of tensioning assembly (500) will be discussed in detail below. Skull clamp (10) is also configured for enhanced imaging. For instance, skull clamp (10) incudes features the promote improved imaging output when skull clamp (10) is being used during imaging. Additional components and operability of the features promoting enhanced imaging will be discussed in detail below.

### I. Exemplary Frame Adjustment Feature

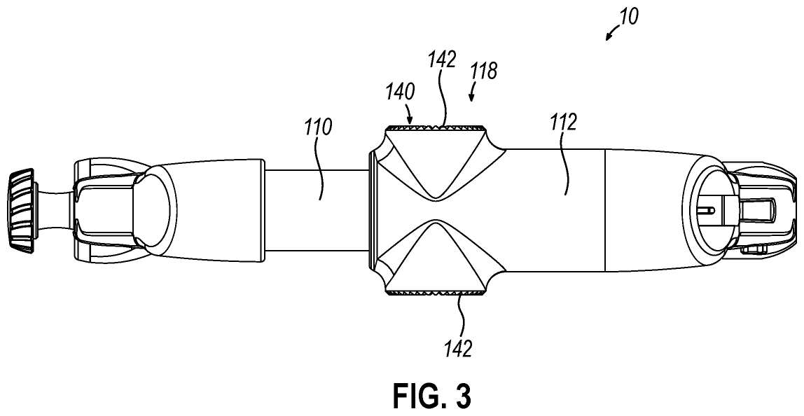

When using skull clamp (10) and stabilizing a patient, at certain times during a procedure it may be necessary or desirable to adjust the spacing or width of frame (100) of skull clamp (10) to accommodate the patient's head. At the same time, it may be necessary or desirable to prevent inadvertent spacing or width adjustment. Referring to FIGS. 1-3, skull clamp (10) comprises frame (100) that has first and second frame portions (102, 104). In some instances, first and second frame portions (102, 104) may be referred to as first and second arms. First and second frame portions (102, 104) each define respective lateral portions (110, 112) and upright portions (114, 116). Upright portions (114, 116) extend away from their respective lateral portions (110, 112). Furthermore, lateral portions (114, 116) collectively define a base (118) of skull clamp (10), and base (118) defines a longitudinal axis (LA1). As will be discussed further below, lateral portions (114, 116) are movable relative to one another along the longitudinal axis (LA1) to adjust the spacing or width of frame (100) of skull clamp (10).

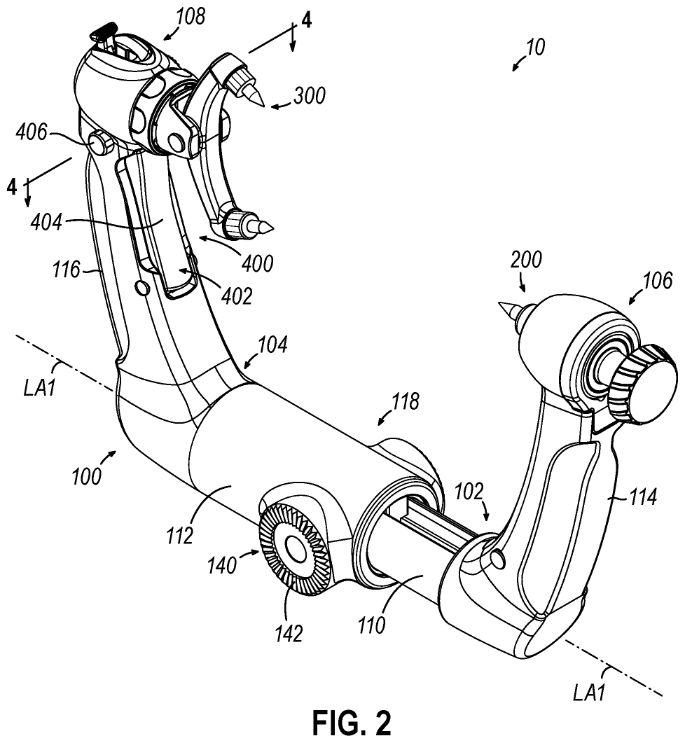

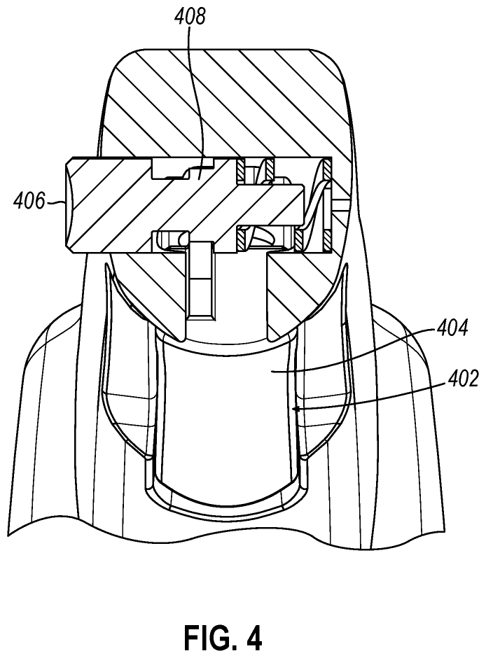

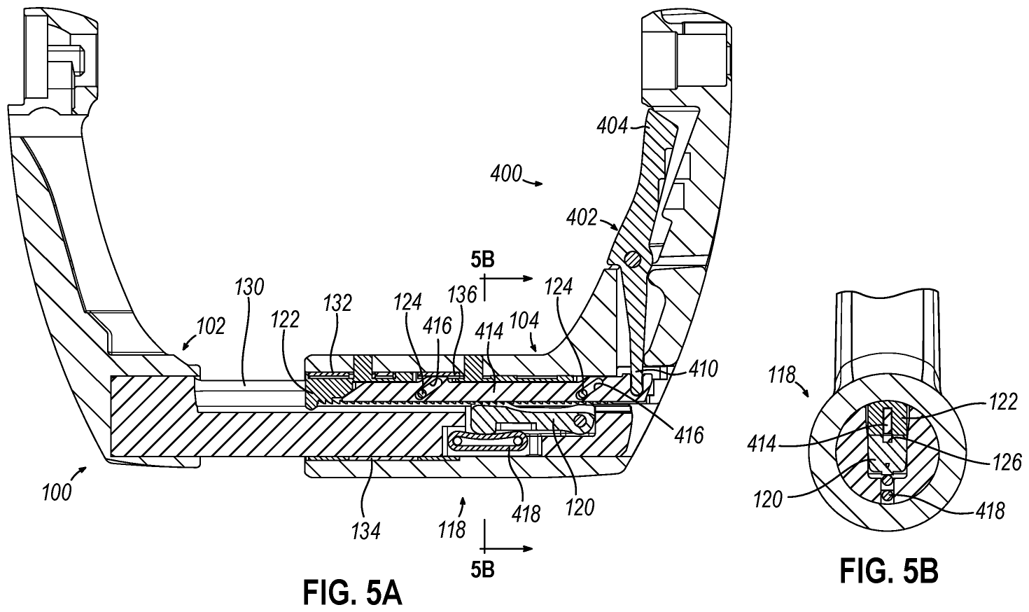

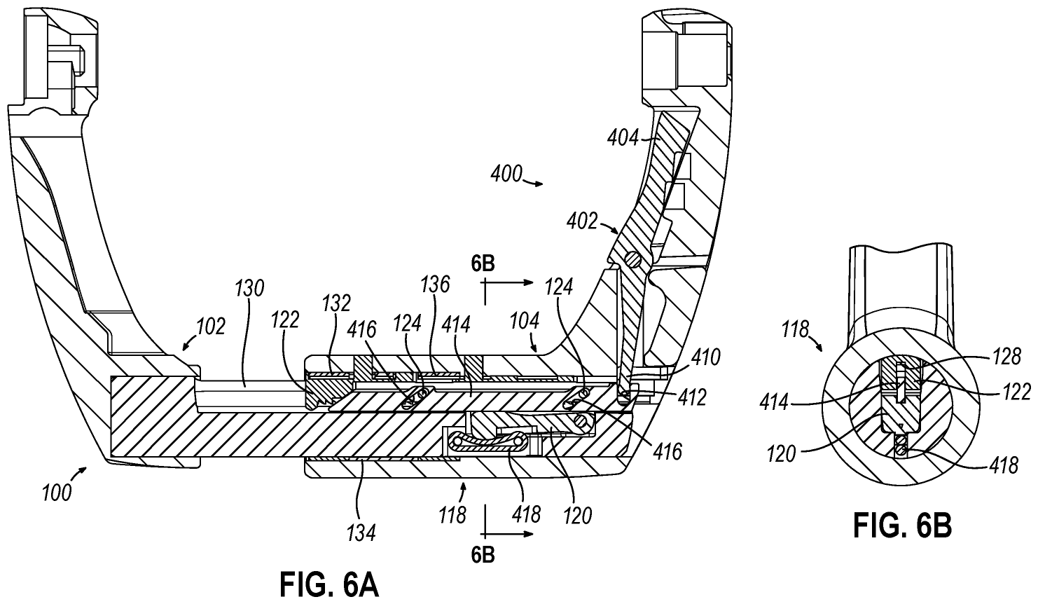

Referring to FIG. 2, skull clamp (10) includes frame adjustment feature (400). Frame adjustment feature (400) includes an actuator assembly (402) having an upper portion (404) that is accessible to the user along upright portion (116) of frame (100). Actuator assembly (402) further comprises a lower portion (414) as seen in FIGS. 5 and 6 as will be discussed further below. As also seen in FIG. 2, a safety release (406) is located along upright portion (116) near or proximate to stabilization assembly (300). Referring to FIG. 4, depressing safety release (406) moves a blocking member (408) of safety release (406) such that it no longer creates an interference with upper portion (404) of actuator assembly (402). In this manner, upper portion (404) of actuator assembly (402) can be actuated or depressed. When safety release (406) is not depressed, it is spring biased so that blocking member (408) contacts upper portion (404) of actuator assembly (402) thereby preventing actuation or depressing upper portion (404) of actuator assembly (402).

Referring to FIG. 5, skull clamp (10) is shown in a locked state in terms of the ability to increase the spacing or width of frame (100). In the present example, in this configuration first and second frame portions (102, 104) can be moved closer together to reduce the spacing or the width of frame (100) and thus skull clamp (10). However, first and second frame portions (102, 104) are locked or prevented from moving apart and thereby the width of frame (100) and thus skull clamp (10) cannot be increased. More specifically, with reference to FIG. 5, first frame portion (102) of skull clamp (10) includes a first toothed engaging member (120), and second frame portion (104) of skull clamp (10) includes a second toothed engaging member (122). The engagement of first and second toothed engaging members (120, 122) prevents increasing the width or spacing of skull clamp (10).

Referring to FIG. 6, skull clamp (10) is shown in an unlocked state in terms of the ability to increase the spacing or width of frame (100) and thus skull clamp (10). In the present example, in this configuration first and second frame portions (102, 104) can be moved closer together to reduce the spacing or the width of frame (100) and thus skull clamp (10). Furthermore, first and second frame portions (102, 104) are unlocked such that they can be moved apart and thereby increase the width of frame (100) and thus skull clamp (10). More specifically, with reference to FIG. 6, actuation of actuator assembly (402) causes disengagement of first and second toothed engaging members (120, 122), which then allows for adjustment of the relative position of first and second frame portions (102, 104) to space them further apart from one another.

Still referring to FIG. 6, in the present example, actuating actuator assembly (402) includes depressing upper portion (404) located along upright portion (116) of second frame portion (104). Upper portion (404) of actuator assembly (402) has a pinned connection with second frame portion (104) at one location such that depressing upper portion (404) causes rotation about the pinned connection.

As mentioned, actuator assembly (402) comprises lower portion (414), which is in mechanical communication with upper portion (404) of actuator assembly (402). More specifically, upper portion (404) includes an end (410) that is received within a slot (412) of lower portion (414). In this configuration, upper portion (404) and lower portion (414) of actuator assembly (402) are in mechanical communication such that rotation of upper portion (404) about its pinned connection with frame (100) causes end (410) to act on lower portion (414). In particular, when actuating actuator assembly (402), end (410) of upper portion (404) pushes lower portion (414). Because lower portion (414) includes diagonal slots (416) and connects with second frame portion (104) via pins (124) received within slots (416), as lower portion (414) is pushed by upper portion (404) lower portion (414) moves in a downward diagonal manner. When actuator assembly (402) is released, the movement of upper portion (404) and lower portion (414) of actuator assembly (402) is opposite from that described above. In particular lower portion (414) moves diagonally upward.

Referring to FIGS. 5B and 6B, when actuator assembly (402) is not actuated, first and second toothed engaging members (120, 122) are engaged as shown in FIG. 5B. When actuator assembly (402) is actuated, first and second toothed engaging members (120, 122) are disengaged as shown in FIG. 6B. As further seen in comparing FIGS. 5B and 6B, when actuator assembly (402) is actuated, first and second toothed engaging member (120, 122) are disengaged by lower portion (414) of actuator assembly (402) pushing downward on first toothed engaging member (120) to separate its toothed portion from the toothed portion of second toothed engaging member (122). To facilitate this engaging and disengaging of first and second toothed engaging members (120, 122), each includes a recess (126, 128) to accommodate lower portion (414) of actuator assembly (402). In the present example, recesses (126, 128) are located within a middle section of respective first and second toothed engaging members (120, 122).

As also shown in FIGS. 5A-6B, frame adjustment feature (400) includes a resilient member (418) that contacts first toothed engaging member (120). Resilient member (418) is configured to bias first toothed engaging member (120) into contact with second toothed engaging member (122). However, when actuator assembly (402) is actuated and lower portion (414) of actuator assembly (402) contacts first toothed engaging member (120) as described above, this contact overcomes the bias of resilient member (418) causing it to elastically deform and thereby allow first toothed engaging member (120) to disengage from second toothed engaging member (122). When actuator assembly (402) is released, resilient member (418) again biases first toothed engaging member (120) into contact and engagement with the second toothed engaging member (122).

Another aspect of frame adjustment feature (400) involves skull clamp (10) having movable components of frame adjustment feature (400) in each of first frame portion (102) and second frame portion (104). As explained above, first frame portion (102) includes first toothed engaging member (120). First toothed engaging member (120) is rotatable about a pinned connection with first frame portion (102) and is movable in response to being contacted by lower portion (414) of actuator assembly (402), or in response to removal of contact with lower portion (414) and contact with resilient member (418). Moreover, actuator assembly (402), and in particular lower portion (414) of actuator assembly (402) is connected with second frame portion (104). In this manner, when unlocking skull clamp (10) to increase the width of skull clamp (10) by increasing the distance between first and second frame portions (102, 104), skull clamp (10) includes moveable components in both arms or portions (102, 104) of skull clamp (10). In use, this configuration can provide for a smoother width adjustment compared to configurations with one arm or portion of the frame having only static components and the other having all the movable components.

Referring again to FIGS. 5A and 6A, first frame portion (102) includes lateral portion (110). In some instances, lateral portion (110) is configured as a guide sleeve or guiding sleeve. In this manner, lateral portion (110) comprises a slot (130) configured to receive second toothed engaging member (122). As mentioned above, also connected with lateral portion (110) is first toothed engaging member (120) as well as resilient member (418).

Second frame portion (104) includes lateral portion (112). Second toothed engaging member (122) has a pinned connection with lateral portion (112). Additionally, lower portion (414) of actuator assembly (402) has a pinned connection with second toothed engaging member (122) via pins (124) and slots (416) described above. In this manner, lower portion (414) of actuator assembly (402) is at least indirectly connected with lateral portion (112) as also further described above.

Lateral portion (112) further includes sleeves or ring members (132, 136) that extend over and around second toothed engaging member (122). Between ring members (132, 136) is a separator (134) that maintains ring members (132, 136) at a predetermined spacing apart from one another. In the present version, ring members (132, 136) are configured to control the contact degree between lateral portions (110, 112). In this manner, ring members (132, 136) act as spacers or glide bushings. As shown, lower portions of ring members (132, 136) are located at the interface of lateral portions (110, 112). In some versions, one or more of ring members (132, 134, 136) can be made from materials that provide a lubricating effect or promote low friction and ease of sliding lateral portions (110, 112) relative to one another. Again, in this manner ring members (132, 136) can be considered bushings or glide bushings that promote gliding or sliding of lateral portions (110, 112) relative to one another. Still in some versions, ring members (132, 136) are considered sleeves or gliding sleeves that facilitate sliding movement between lateral portions (110, 112). While the illustrated version shows two ring members (132, 136), in other versions there may be a greater or fewer number of ring members.

Additionally associated with lateral portion (112) is a pair of raised features (138) as seen in FIGS. 5A and 6A. Raised features (138) are located along a top of second toothed engaging member (122) and are positioned such that they are in contact with an inner surface or interior wall of lateral portion (112). Similar to ring members (132, 136), raised features (138) are configured to control or define the contact amount or degree between lateral portions (110, 112) albeit in this case indirectly based on raised features (138) controlling the contact degree between the inner surface or interior wall of lateral portion (112) and second toothed engaging member (122). In some versions, ring members (132, 136) and raised features (138) are configured to collectively or cooperatively promote ease of sliding or moving lateral portions (110, 112) relative to each other by controlling the amount or degree of contact between lateral portions (110, 112). This control of contact amount can also be considered to be controlling an amount of friction between the components.

### II. Exemplary Base Configuration for Improved Imaging

As mentioned above, many procedures use imaging with various modalities like MRI, CT scans, X-ray, etc. A concern when imaging a patient stabilized with a skull clamp is to minimize image artifacts that appear in the imaging output. Type and amount of material can be factors that influence image artifacts. Skull clamp (10) incorporates these and other features that help minimize image artifacts and improve the overall quality of the imaging output. As will be described below, some of these other features include a curved shape for base (118) about longitudinal axis (LA1) defined by base (118), and in particular the curved shape located along a top region and a bottom region along a length of base (118).

Referring to FIGS. 1-3, skull clamp (10) comprises base (118) as described above. As shown in the illustrated example, base (118) is generally cylindrical in shape. The terms “generally” here meaning not entirely cylindrical along its entire length. Furthermore, base (118) is formed by lateral portions (110, 112) of respective first and second frame portions (102, 104). In this manner, lateral portion (110) with a generally cylindrical shape is received within an opening in lateral portion (112). Furthermore, lateral portions (110, 112) are slidable relative to one another in a selective locking fashion to change a width or spacing of skull clamp (10) as described above. In some versions, lateral portion (110) is telescopic with lateral portion (112). In some versions, lateral portions (110, 112) are concentrically arranged.

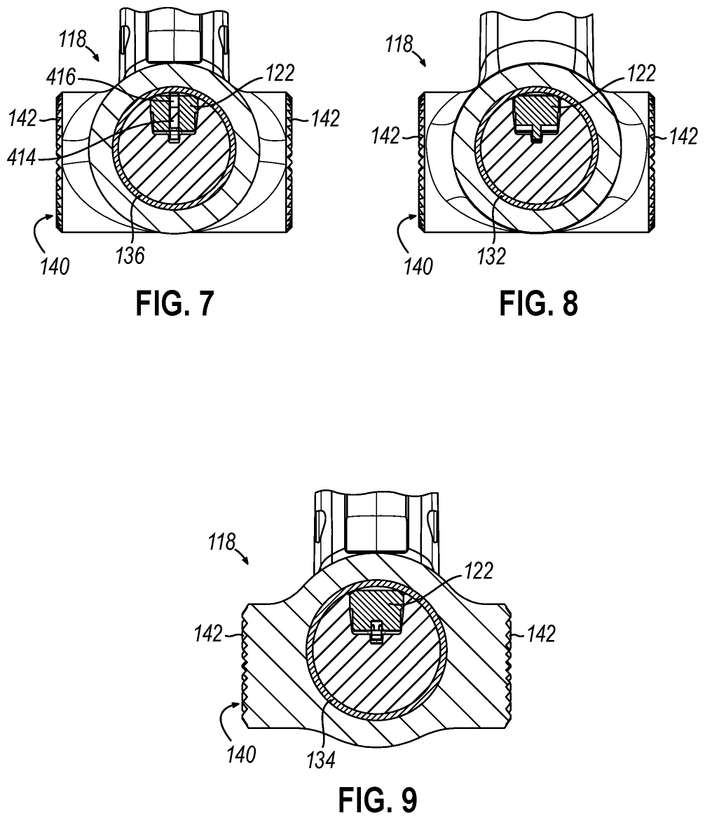

Referring to FIGS. 7-9, base (118) includes interface (140) that has engaging features (142) facing both a front and a rear of skull clamp (10). In this manner, with base (118) defining longitudinal axis (LA1) as described above, interface (140) is oriented transverse to longitudinal axis (LA1) such that engaging features (142) of interface (140) are oriented transverse to longitudinal axis (LA1). With this configuration, skull clamp (10) includes the generally cylindrically shaped base (118) defining longitudinal axis (LA1), and with base (118) including interface (140) oriented transverse to longitudinal axis (LA1).

When referring to FIGS. 7 and 8, skull clamp (10) in the present example includes base (118) and interface (140) as mentioned. As shown, on each side of interface (140) base (118) has a circular shape in cross section along at least a portion of its length. Referring to FIG. 9, at the location along base (118) having interface (140), base (118) has a curved shape in cross section along a top region and a bottom region of base (118). Additionally at this location, base (118) has a serrated shape in cross section along a front region and a rear region of base (118).

### III. Exemplary Tensioning Assembly and Adjustment

When a head fixation device such as skull clamp (10) is used to stabilize a patient, one part of the stabilization is to adjust the pressure being applied to the patient's head. One way this is done is to adjust the amount of force the stabilization assembly applies to the connected one or more stabilizing features that contact the patient's head. The following paragraphs will describe exemplary tensioning assembly (500) that is used to adjust the force applied to the stabilizing feature(s) and thus adjust the pressure at the patient's head.

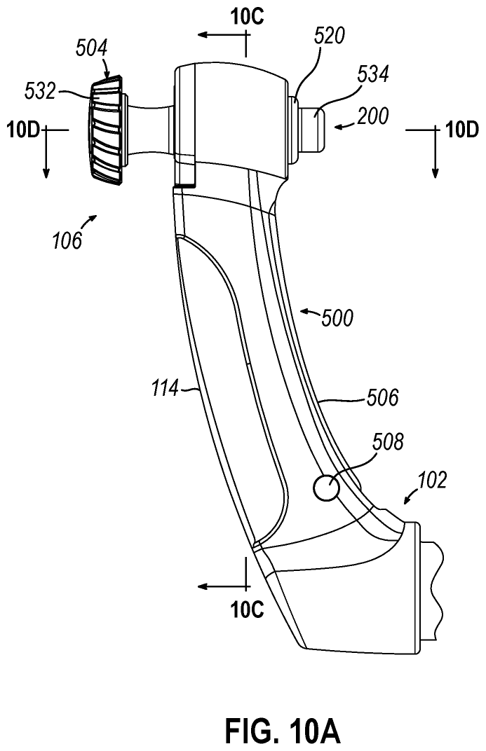

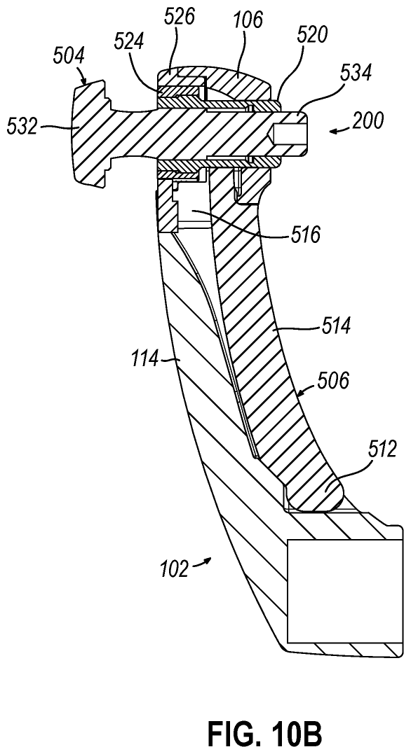

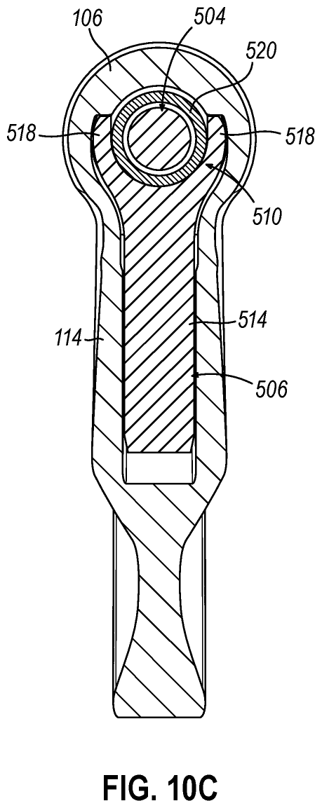

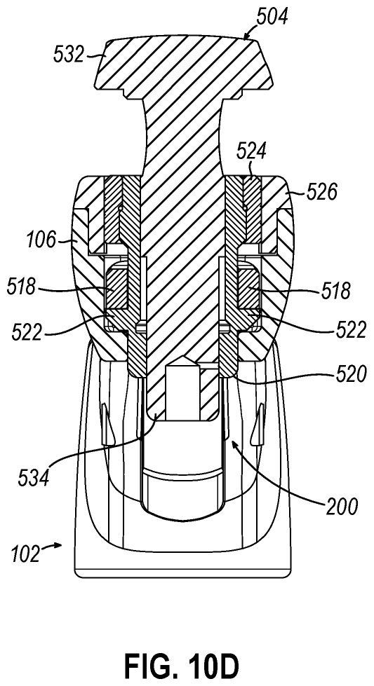

Referring to FIGS. 1 and 10A, tensioning assembly (500) is shown housed at least in part within upright portion (114) of first frame portion (102). More specifically, tensioning assembly (500) comprises a stabilization assembly (200), an actuator (504), and a tensioning feature (506). In the present example the tensioning feature (506) may also be referred to as a bending beam.

FIG. 10B illustrates a cross section view of tensioning assembly (500). Tensioning feature (506) connects with first frame portion (102). In the present example, tensioning feature (506) is pinned with first frame portion (102) by pin (508) as seen in FIG. 10A. Tensioning feature (506) includes a first end (510) and a second end (512) with a middle section (514) extending between first end (510) and second end (512). As shown in FIG. 10B, near second end (512), first frame portion (102) contacts tensioning feature (506), while near first end (510) along an upper region of tensioning feature (506) there is a gap or space (516) between first frame portion (102) and tensioning feature (506). With this configuration, tensioning feature (506) is prevented from rotating about pin (508) based on the impinging or contacting first frame portion (102) on the lower region of tensioning feature (506). Referring to FIG. 10C, first end (510) of tensioning feature (506) has a split end configuration with a pair of extensions (518) separated by a space. In some instances, pair of extensions (518) may be referred to as a pair of arms or a pair of contacts.

Referring now also to FIGS. 10C and 10D, at first end (510) tensioning feature (506) contacts a portion of stabilization assembly (200). As best seen in FIG. 10D, stabilization assembly (200) includes a threaded sleeve (520) that has a pair of protrusions (522). Pair of extensions (518) are located proximally of pair of protrusions (522). Stabilization assembly (200) further includes a calibration sleeve (524) that is threadably connected with threaded sleeve (520). Stabilization assembly (200) further includes a centering ring (526) that is configured to receive calibration sleeve (524). In the present example, calibration sleeve (524) and centering ring (526) have a keyed arrangement such that calibration sleeve (524) can translate relative to centering ring (526) when received by centering ring (526), but calibration sleeve (524) cannot rotate relative to centering ring (526) when calibration sleeve (524) is received by centering ring (526). In the present example, calibration sleeve (524) includes notches (528) about its circumferential outer surface, while centering ring (526) includes a complementary protrusion about its inner surface that is receivable within one of notches (528). In this manner calibration sleeve (524) can be received by centering ring (526) in multiple rotational orientations or positions.

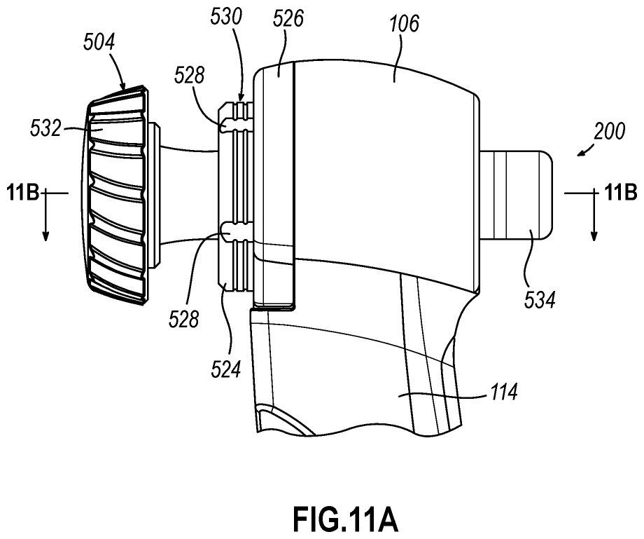

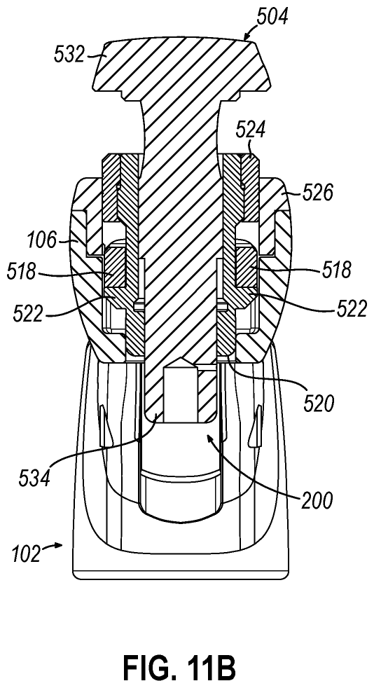

As shown in FIG. 11A, calibration sleeve (524) further includes markings, engravings, or other indicating features (530) that indicate a force amount that correlates to the force being applied by the one or more stabilizing features to the head of the patient. In some versions, indicating feature (530) is a force gauge. In one example, notches (528) and indicating features (530) are arranged such that locating the protrusion of centering ring (526) with different notches (528) when locating calibration sleeve (524) within centering ring (526) orients indicating features (530) differently thereby allowing this to operate as a calibration structure for tensioning assembly (500).

As mentioned above, receiving portion (106) of skull clamp (10) is configured to receive a stabilization assembly, and in the present example, stabilization assembly (200). In one example, receiving portion (106) is configured with a keyed orientation with threaded sleeve (520) such that threaded sleeve (520) can translate relative to receiving portion (106) but is prevented from rotating relative to receiving portion (106).

Tensioning assembly (500) further includes actuator (504). Actuator (504) threadably connects with threaded sleeve (520). In the present example, actuator (504) is located at an upper portion of skull clamp (10) and is also co-axial with stabilization assembly (200). Actuator (504) includes a first end (532) that in one example is configured to be grasped for rotating actuator (504). Actuator (504) also includes a second end (534) that is configured to receive one or more stabilizing features, which may be in the form of pins or pads. In one example as shown in FIG. 1, second end (534) receives a skull pin, while the opposing stabilization assembly (300) includes a rocker arm assembly having two skull pins. In this configuration, a three-point fixation or stabilization is achieved. In some other versions a rocker arm assembly the same or similar to that shown in FIGS. 1-2 is used in place of the single pin with stabilization assembly (200). In this configuration there are four pins on each side and thus a four-point fixation or stabilization is achieved.

When using tensioning assembly (500) to adjust a stabilization force applied to the patient, an initial setup is used as shown in FIGS. 10A-10D where calibration sleeve (530) is flush with a proximal side of receiving portion (106). In this particular example, such a configuration equates generally to 0 newtons stabilizing force. After skull clamp (10) width has been adjusted such that the stabilizing features are in contact with the patient, actuator (504) is rotated to apply the desired amount of stabilizing force, sometimes referred to as a pinning force where the stabilizing features are pins. Based on the contact of the stabilizing features with the patient and the threaded connection between actuator (504) and threaded sleeve (520), when rotating actuator (504), threaded sleeve (520) translates proximally away from the patient and stabilizing features. At the same time, calibration sleeve (524) translates with threaded sleeve (520) of stabilization assembly (200) based on the threaded engagement of calibration sleeve (524) with threaded sleeve (520).

As best seen when comparing FIG. 10D to FIG. 11B, as threaded sleeve (520) translates proximally, protrusions (522) push on extensions (518) of first end (510) of tensioning feature (506) causing a deformation or bending of first end (510). This deformation or bending can occur in part because the second end (512) of tensioning feature (506) is fixed relative to first frame portion (102) but space (516) exists proximal of first end (510) of tensioning feature (506). The bending or deformation of tensioning feature (506) creates a bending moment that applies a force distally towards the patient. More specifically, this bending moment and the force from it are transmitted to the one or more stabilizing features retained by second end (534) of actuator (504), with the one or more stabilizing features being in contact with the patient. This increases the stabilizing force applied and thus the stabilizing pressure at the location where the stabilizing feature(s) contact the patient. Based on the proximal movement of calibration sleeve (524), markings or indicating features (530) are revealed and correspond with the amount of force being applied.

In view of the above description and drawings, it has been shown that skull clamp (10) for stabilizing a patient using two or more stabilizing features configured to contact the patient comprises frame (100), stabilization assembly (200) connectable with frame (100), tensioning feature (506), and actuator (504). Tensioning feature (506) comprises an elongated member having first end (510), second end (512), and middle section (514) extending between first end (510) and second end (512), wherein first end (510) is configured to contact a portion of stabilization assembly (200) and second end (512) is configured to be fixed, directly or indirectly, with frame (100). Actuator (504) is configured to adjust an amount of a first force applied by the stabilization feature to the patient, wherein actuating actuator (504) causes the portion of stabilization assembly (200) to apply a second force on tensioning feature (506) which produces a bending moment in tensioning feature (506) that imparts the first force at first end (510) of tensioning feature (506) onto the portion of stabilization assembly (200) in a direction towards the patient.

In a similar manner, it has been shown that skull clamp (10) for stabilizing a patient using two or more stabilizing features configured to contact the patient comprises frame (100), stabilization assembly (200) connectable with frame (100), tensioning feature (506), and actuator (504). Tensioning feature (506) comprises an elongated member having first end (510), second end (512), and middle section (514) extending between first end (510) and second end (512), wherein first end (510) is configured to contact a portion of stabilization assembly (200) and second end (512) is configured to be fixed, directly or indirectly, with frame (100). Actuator (504) is configured to translate a portion of stabilization assembly (200), wherein translation of the portion of stabilization assembly (200) bends first end (510) of the elongated member producing a bending moment in tensioning feature (506) imparting a first force in a direction towards the patient.

In also a similar manner, it has been shown that skull clamp (10) for stabilizing a patient using two or more stabilizing features configured to contact the patient comprises frame (100), stabilization assembly (200) connectable with frame (100), tensioning feature (506), and actuator (504). Tensioning feature (506) comprises an elongated member having first end (510), second end (512), and middle section (514) extending between first end (510) and second end (512), wherein first end (510) is configured to contact a portion of stabilization assembly (200) and second end (512) is configured to be fixed, directly or indirectly, with frame (100). Actuator (504) is configured to bend first end (510) of the elongated member producing a bending moment in tensioning feature (506) imparting a first force in a direction towards the patient.

### IV. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A skull clamp for stabilizing a patient comprising a frame having a base defining a length and a longitudinal axis extending along the length between a first upright portion and a second upright portion, wherein the base comprises a generally cylindrical shape having a circular shape in a cross section taken transverse to the longitudinal axis along at least a portion of the length.

2. The skull clamp of claim 1, wherein the base further comprises an interface configured to attach the skull clamp, directly or indirectly, with a patient support structure.

3. The skull clamp of claim 1, wherein the first and second upright portions each are configured to receive a stabilization assembly configured to retain one or more stabilizing features configured to contact a head of the patient.

4. The skull clamp of claim 2, wherein the interface is oriented transverse to the longitudinal axis.

5. The skull clamp of claim 1, wherein a width of the frame is adjustable based on a relative position of the first and second upright portions with respect to each other.

6. The skull clamp of claim 1, wherein the base comprises a first substantially cylindrical portion and a second substantially cylindrical portion, wherein the first substantially cylindrical portion and the second substantially cylindrical portion are adjustable by the sliding relative to each other.

7. The skull clamp of claim 6, wherein the base comprises one or more spacers configured to maintain a spacing between the first and second substantially cylindrical portions while also reducing a surface area of contact between the first and second substantially cylindrical portions.

8. A skull clamp for stabilizing a patient using two or more stabilizing features configured to contact the patient, the skull clamp comprising:
(a) a frame;
(b) a stabilization assembly connectable with the frame;
(c) a tensioning feature comprising an elongated member having a first end, a second end, and a middle section extending between the first end and the second end, wherein the first end is configured to contact a portion of the stabilization assembly and the second end is configured to be fixed, directly or indirectly, with the frame; and
(d) an actuator configured to adjust an amount of a first force applied by the stabilization feature to the patient, wherein actuating the actuator causes the portion of the stabilization assembly to apply a second force on the tensioning feature which produces a bending moment in the tensioning feature that imparts the first force at the first end of the tensioning feature onto the portion of the stabilization assembly in a direction towards the patient.

9. The skull clamp of claim 8, comprising a force gauge configured to indicate the first force applied by the stabilization feature to the patient.

10. The skull clamp of claim 8, wherein the actuator is rotatable in place to adjust the amount of the first force.

11. The skull clamp of claim 8, wherein the first end of the elongated member of the tensioning feature bends in response to the second force imparted on the first end by the portion of the stabilization assembly.

12. The skull clamp of claim 8, wherein the actuator is configured to retain at least one of the two or more stabilizing features.

13. The skull clamp of claim 8, wherein the first end of the tensioning feature comprises a pair of contacts separated by a space therebetween.

14. The skull clamp of claim 8, wherein the first force comprises a distal force extending toward the patient, and wherein the second force comprises a proximal force extending away from the patient.

15. A skull clamp for stabilizing a patient comprising:
(a) a frame having a first arm and a second arm, wherein the first arm and the second arm are selectively engageable, and wherein a width of the frame is adjustable based on a relative position of the first and second arms; and
(b) a locking assembly configured to selectively lock the relative position of the first and second arms to prevent the width of the frame from increasing, wherein the locking assembly comprises:
(i) a first engaging feature connectable with the first arm,
(ii) a second engaging feature connectable with the second arm, and
(iii) an actuator assembly connectable with the first arm, wherein the actuator assembly is moveable relative to the first arm to contact the second engaging feature connectable with the second arm, wherein the second engaging feature is moveable relative to the second arm.

16. The skull clamp of claim 15, wherein the locking assembly further comprises a safety release configured to prevent movement of the actuator assembly when the safety release is in a first engaged position, and configured to allow movement of the actuator assembly when the safety release is in a second disengaged position.

17. The skull clamp of claim 15, wherein a portion of the actuator assembly is positioned along an upright portion of the first arm proximate to a stabilization assembly configured to retain one or more stabilization features for contacting the patient.

18. The skull clamp of claim 15, wherein the first engaging feature is immobile relative to the first arm, and wherein the second engaging feature is movable relative to the first and second arms.

19. The skull clamp of claim 15, wherein the locking assembly further comprises a resilient member configured to impart a bias on the second engaging feature to a locked position.

20. The skull clamp of claim 19, wherein the contact between the actuator assembly and the second engaging feature deforms the resilient member to overcome the bias and move the second engaging feature to an unlocked position.

21. The skull clamp of claim 15, wherein the actuator assembly comprises a first portion configured to rotate relative to the first arm, wherein the actuator assembly comprises a second portion configured to move diagonally relative to the first arm in response to rotation of the first portion.
